(19) United States
(12) Plant Patent Application Publication
(10) Pub. No.: US 2005/0223444 P1
(43) Pub. Date: Oct. 6, 2005

(54) Climber rose plant named 'ADABARING'

(75) Inventor: Michel Adam, Liffre (FR)

Correspondence Address: KLARQUIST SPARKMAN, LLP, 121 SW SALMON STREET, SUITE 1600, PORTLAND, OR, 97204, US

(73) Assignee: LUX RIVIERA S.r.1.

(21) Appl. No.: 11/090,921

(22) Filed: Mar. 24, 2005

(30) Foreign Application Priority Data
Mar. 31, 2004 (EP) 2004/630

(51) Int. Cl. A01H 5/00

(52) U.S. Cl. PLT/115

(57) ABSTRACT

Climber rose plant having vigor; more numerous leaves; darker red color; better climbing-up capability; disease resistance; and strong fragrance.

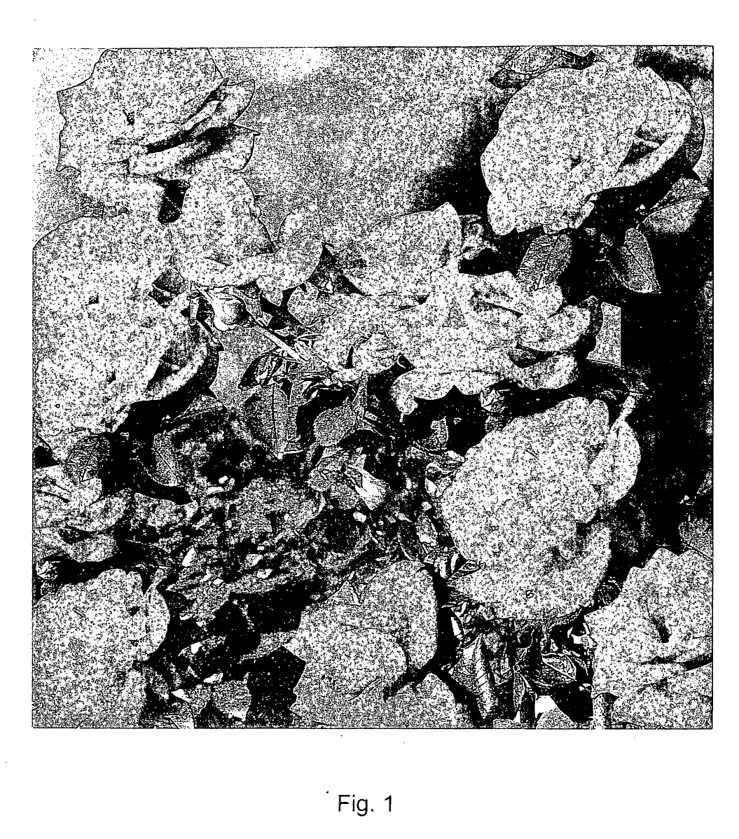

### Genus and Species

Rosa hybrida

### Variety Denomination

‘ADABARING’

### Origin of the Invention

The present invention relates to a new and distinct variety of a climber rose plant named ‘ADABARING’ which was originated by me by crossing the variety ‘DELSIRE’ (unpatented), with the variety ‘MEITULIMON’ (unpatented).

### BACKGROUND OF THE INVENTION

The primary objective of this breeding was to produce a new rose variety having a large double red climbing rose, strong foliage with more vigor and energy, a better climbing-up activity and a darker red color.

### BRIEF SUMMARY OF THE INVENTION

The objective was substantially achieved, along with other desirable improvements, as evidenced by the following unique Combination of characteristics that are outstanding in the. new variety and that distinguish it from its parents, as well as from all other varieties of which I am aware:

1. More vigor.

2. More leaves.

3. Darker red.

4. Better climbing-up capability.

5. Disease resistance to black spot and oidium.

6. Extremely fragrant.

Asexual reproduction of this new variety by grafting onto multiflora (unpatented) rootstock, as performed at Liffre, France, shows that the foregoing and all other characteristics and distinctions come true to form and are established and transmitted through succeeding propagations.

### Comparison with Parents

The female parent ‘DELSIRE’ is less fragrant, has bigger leaves, is less vigorous, and has pink flowers in comparison to ‘ADABARING’. The male parent ‘MEITULIMON’ is less vigorous and has mauve-pink flowers in comparison to ‘ADABARING’.

### Comparison with Similar Variety

Refer to the above comparison with the parent plants.

### BRIEF DESCRIPTION OF THE ILLUSTRATION

The accompanying illustration shows typical specimens of the vegetative growth and flowers of this new variety in different stages of development, depicted in color as nearly true as it is reasonably possible to make the same in a color illustration of this character.

### DETAILED BOTANICAL DESCRIPTION

The following is a detailed description of my new rose cultivar with color descriptions using terminology in accordance with The Royal Horticultural Society (London) Colour Chart (2001), except where ordinary dictionary significance of color is indicated. It should be noted that color is difficult to determine precisely and can vary with growing conditions.
Parentage:
   Seed parent.—‘DELSIRE’.
   Pollen parent.—‘MEITULIMON’.

### Flower

Observations were made over an eight year period. Then following observations are of eight year old plants grown on multiflora rootstock in a garden environment at Liffre, France.
Blooming habit: From May until frost.
Bud:
   Size.—About 16 cm in diameter when the petals start to unfurl.
   Form.—The bud form is pointed ovoid.
   Color.—When sepals first divide, bud color is rouge cardinal (RHS 53A). When half blown, the upper sides of the petals are strong red (RHS 53A), and the lower sides of the petals are strong red (RHS 53A).
   Sepals.—Color: Upper surface: Pale green (RHS 135D). Lower surface: Green (RHS 140A) and Green-Bronze (RHS 149B). Length: 3-4 cm. Width: 1.3-1.5 cm. Shape: Elongated. Surface texture: Smooth. There are three normally appendaged sepals. There are two unappendaged sepals with smooth edges.
   Receptacle.—Color: Green (RHS 141A). Shape: Funnel. Size: Medium, about 1.5 cm long×1.5 cm wide. Surface: Smooth.
   Peduncle.—Length: Long, averaging about 5-8 cm long. Surface: Smooth. Color: Green (RHS 142A) Strength: Stiff, erect.
Bloom:
   Size.—Large. Average open diameter is about 1.3-1.6 cm.
   Borne.—Singly and several together in clusters of 3-5 blooms having rounded shape.
   Stems.—Strength: Normal. Average length is about 30-60 cm with a diameter of 3-5 mm.
   Form.—When first open: High centered. Permanence: Flattens toward the end of the bloom.
   Petalage.—Number of petals under normal conditions: 23-30.
   Color.—The upper sides of the petals are strong red (RHS 53A). The reverse sides of the petals are less strong red (RHS 53A). The base of the petal is a strong red with yellow nuances (RHS 150C). The major color on the upper side is strong red (RHS 53A).
   Variegations.—None observed.
   Discoloration.—The general tonality at the end of the first day is red (RHS 59A). At the end of the third day the general tonality is red (RHS 60A). Further aging: No change.
   Fragrance.—Strong, spicy.
Petals:
   Texture.—Thick.
   Shape.—Oval.
   Surface.—Smooth.
   Margin.—Smooth.
   Apex shape.—Rounded.
   Base shape.—Acute.
   Form.—edges slightly quilled.
   Arrangement: Informal.
   Petaloids: Number: 5 to 10. Color: Red (RHS 59A).
   Persistence.—Petals hang on and dry.
   Lastingness.—On the plant: Long. As a cut flower: Not tested.
Reproductive parts:
   Stamens.—Number: Many. Length: Normal. Anthers: Length: Normal. Color: Yellow (RHS 22B) sometimes with a little red (RHS 42A). Arrangement: Regular around styles. Filaments: Color: Red (RHS 42A). Length: 1.4 cm Pollen: Color: Yellow (RHS 22C).
   Pistils.—Number: 60. Length: 0.5 to 0.8 cm. Styles: Color: Cream (RHS 42A). Length: Normal. Stigmas: Color: Cream (RHS 22C). Length: Normal.
   Hips.—Color: Red (RHS 24A). Size: 2.3 cm.

### Plant

Form.—Climber.
Growth.—Very vigorous.
Age at maturity.—3 years.
Mature plant.—Height: Three to four meters. Width: Often 150 cm to 180 cm.
Rootstock.—Multiflora.
Foliage: Number of leaflets on normal mid-stem leaves: 5.
   Size: Medium. About 5 cm long and 7/10 wide.
   Quantity: Abundant.
   Color: New foliage: Upper side: Brown Bronze (RHS 136A). Lower side: Brown Red (RHS 130B). Old foliage: Upper side: Brilliant Green (RHS 139A). Lower side: Green (RHS 139B).
Leaflets:
   Size.—About 6 cm long×3 cm wide.
   Shape.—Pointed oval.
   Base shape.—Acute.
   Texture.—Leathery.
   Edge.—Serrated.
   Serrations.—Single, small.
   Petiole.—Color: Pale green (RHS 141A).
   Petiole rachis.—Color: Base, clear green (RHS 141D). Underside: With prickles.
   Auricles.—None.
   Stipules.—Length: 1 cm, smooth.
   Disease resistance.—Resistant to mildew, resistant to rust and resistant to blackspot under normal growing conditions.
   Pest resistance.—Good resistance to pests.
Wood:
   New wood.—Color: Green (RHS 136B). Bark: Smooth.
   Old wood.—Color: Green Bronze (RHS 139A). Bark: Rough.
Prickles:
   Quantity.—On main canes from base: Few. 10-15 per 6 cm of cane length. On laterals from main canes: Few.
   Form.—Hooked downward.
   Length.—0.3 to 0.5 cm.
   Color when young.—Red bronze (RHS 181B).
   Color when mature.—Red (RHS 182B).
Small prickles:
   Quantity.—On main stalks: Few. On laterals: Few.
   Color.—When young: Red Bronze (RHS 179A). When mature: Red (RHS 180B).
Winter hardiness: Not tested.

1. A new and distinct variety of rose plant of the climber class, substantially as herein shown and described, characterized particularly by its vigor; more numerous leaves; darker red color; better climbing-up capability; disease resistance; and strong fragrance.
